(19) United States
(12) Patent Application Publication
(10) Pub. No.: US 2026/0000877 A1
(43) Pub. Date: Jan. 1, 2026

(54) FLUID ADMINISTRATION DEVICE

(71) Applicant: Astute MedTech, LLC, Morrisville, PA (US)

(72) Inventors: Benjamin M. Davis, Woodstock, GA (US); Marc S. Waldman, Yardley, PA (US); Lillian Sheedy, St. Petersburg, FL (US); Carine Henderson, Sandy, UT (US)

(21) Appl. No.: 19/252,523

(22) Filed: Jun. 27, 2025

Related U.S. Application Data
(60) Provisional application No. 63/665,678, filed on Jun. 28, 2024.

(51) Int. Cl. A61M 31/00; A61M 5/28; A61M 39/00; A61M 39/10

(52) U.S. Cl. CPC A61M 31/002; A61M 5/288; A61M 39/10; A61M 2039/0009; A61M 2202/0007; A61M 2202/04; A61M 2205/273; A61M 2205/584; A61M 2207/00; A61M 2209/06; A61M 2210/0625

(57) ABSTRACT

A fluid administration device is disclosed. According to one example implementation, the fluid administration device includes a connector, a tube, and an applicator. The connector is configured to connect to a corresponding device, such as a syringe or other vessel filled with medication, colostrum, or other fluid. A proximal end of the tube can be affixed to the connector. A distal end of the tube can be connected to the applicator. Fluid from the corresponding device can flow into the fluid administration device via the connector, into the tube, and into the foam applicator. The applicator can be inserted into the buccal cavity of a patient and massaged along the buccal mucosa to administer the fluid.

### CROSS-REFERENCE TO RELATED APPLICATIONS

This application claims the benefit of and priority to U.S. Provisional Application Ser. No. 63/665,678, filed Jun. 28, 2024, titled “FLUID ADMINISTRATION DEVICE,” the entire contents of which is hereby incorporated by reference.

### BACKGROUND

Administration of oral liquid medications, nutritionals, and other fluids for infants, children, and individuals with limited faculties poses significant challenges in ensuring accurate dosing due to their inability to communicate or cooperate fully. Oral methods like bottles, sippy cups, syringes, and droppers often fail to guarantee precise fluid intake, risking under- or over-dosing. Patients can easily spit up fluids that are administered via these methods and this can create an underdosing situation which a caregiver can then try to compensate for but in doing so creates an overdosing situation. There is a need for a device that addresses these issues to ensure the safe administration of medications while preventing medical tubing misconnections and promoting patient safety.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1A is a schematic diagram depicting an orthographic side view of a fluid administration device according to an example implementation.

FIG. 1B is a schematic diagram depicting another orthographic side view of a fluid administration device according to an example implementation.

FIG. 1C is a schematic diagram depicting a perspective side view of a fluid administration device according to an example implementation.

FIG. 1D is a schematic diagram depicting another perspective side view of a fluid administration device according to an example implementation.

FIG. 1E is a schematic diagram depicting an orthographic rear view of a fluid administration device according to an example implementation

FIG. 1F is a schematic diagram depicting an orthographic front view of a fluid administration device according to an example implementation.

FIG. 2 is a flow diagram depicting a method for assembling a fluid administration device according to an example implementation.

FIG. 3 is a flow diagram depicting a method for using a fluid administration device according to an example implementation.

FIG. 4 is a flow diagram depicting a method for prophylactically administering colostrum via a fluid administration device according to an example implementation.

### DETAILED DESCRIPTION

Device misconnections in medical settings can lead to severe and sometimes fatal consequences, particularly when it involves the administration of enteral fluids, medications, or nutrients. Traditional connectors can inadvertently be attached to non-enteral devices, resulting in the inadvertent administration of fluids into the wrong area of therapy, which can cause serious health issues. Additionally, existing methods of buccal administration for fluids often lack control, leading to inefficient absorption, patient discomfort, and increased risk of contamination.

The present disclosure is directed to a fluid administration device that addresses these issues by incorporating a connector on one end and a foam applicator on the other end. The connector ensures compatibility with designated enteral devices, thereby preventing misconnections. The foam applicator allows for the controlled and hygienic administration of fluid directly onto the buccal mucosa, enhancing absorption and effectiveness of the fluid.

Current approaches include traditional connectors that can sometimes be mistakenly attached to non-enteral devices, leading to harmful misconnections. Additionally, manual swabbing or finger administration in current buccal administration methods, where swabs or a caregiver's finger are manually wetted with medication or fluid, can be inefficient and increase the risk of contamination. The use of a syringe may provide an accurate amount of fluid, but administration using a syringe may be difficult for patients that cannot swallow properly or are prone to spitting up. Direct oral administration methods, where fluids are directly administered into the mouth without controlled administration, lead to potential spillage, patient discomfort, and inconsistent absorption.

The disclosed fluid administration device offers several advantages over these prior solutions. The connector ensures the disclosed device cannot be mistakenly connected to non-enteral devices, significantly reducing the risk of misconnections and associated complications. The foam applicator provides a controlled release of fluids, ensuring consistent and efficient absorption through the buccal mucosa. This controlled mechanism is particularly beneficial for medications requiring rapid onset, such as glucose gel for treating hypoglycemia, and the administration of colostrum to premature infants prophylactically against late onset sepsis (LOS), necrotizing enterocolitis (NEC), and ventilator associate pneumonia.

The disclosed fluid administration device also enhances patient safety and comfort with a pliable tube and non-shedding, porous substrate applicator (e.g., foam), both designed to prevent injury and discomfort during use. The disclosed fluid administration device is also designed to reduce the risk of the patient spitting out the administered fluid, ensuring more effective administration. The disclosed fluid administration device can be individually packaged for single-use to reduce the risk of contamination compared to manual swabbing methods. The semi-closed system further supports infection prevention initiatives.

By bypassing gastrointestinal degradation and the hepatic first-pass effect, the disclosed fluid administration device allows for better bioavailability of medications and therapeutic substances, leading to more rapid and effective treatment outcomes. The fluid administration device is versatile and suitable for various clinical scenarios, including emergency situations where patients are unresponsive or unconscious. The fluid administration device also provides a solution for administering colostrum and other therapeutic substances to neonates and other vulnerable populations. Additionally, the design of the fluid administration device allows for straightforward connection to compatible devices, such as syringes (e.g., ENFit® type), bulbs, ampules, and other vessels for controlled administration of fluid. This makes the fluid administration device easy for healthcare providers to administer medications accurately and efficiently. Thus, the disclosed fluid administration device provides significant advancements in patient care and safety by addressing the limitations of existing methods and offering a comprehensive solution for safe, controlled, and effective buccal administration of fluids.

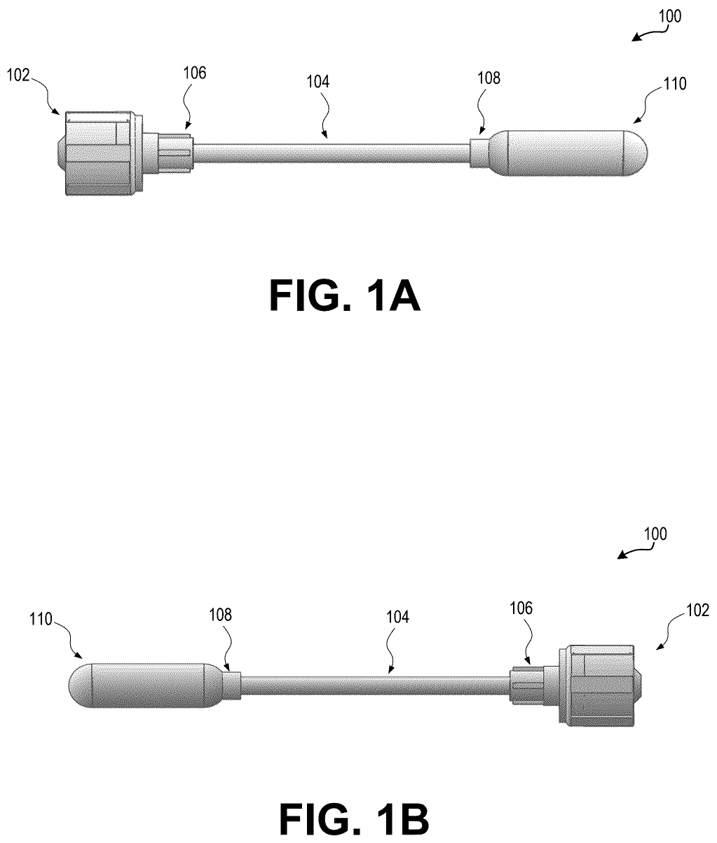

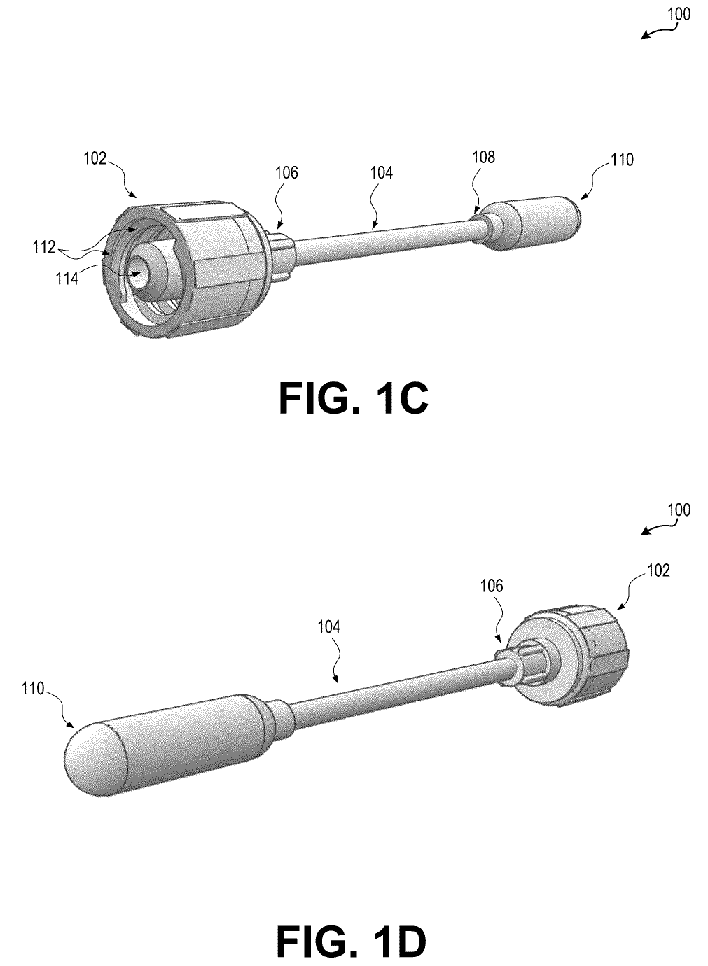

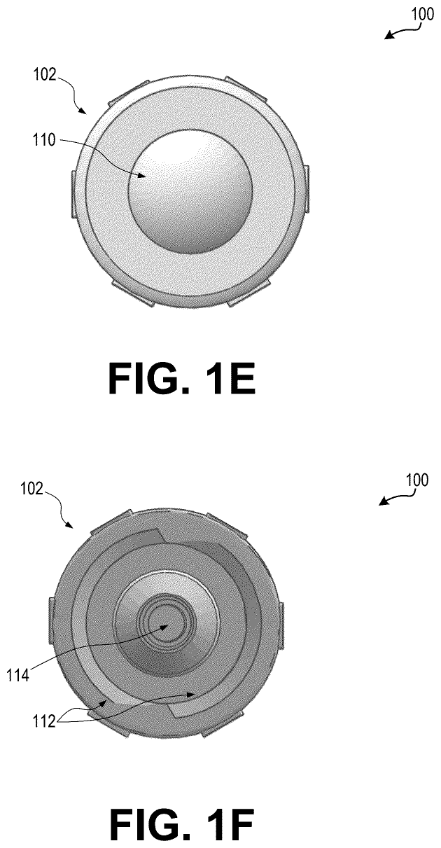

Turning now to FIG. 1A, shown is a schematic diagram depicting an orthographic side view of a fluid administration device 100 according to an example implementation. The fluid administration device 100 can be provided as a single unit that includes multiple components connected together, such as during manufacture. Alternatively, the fluid administration device 100 can be provided as two or more separate components that are usable together to form the fluid administration device 100.

The fluid administration device 100 includes a connector 102. The connector 102 is a type of connector that can be used with compatible devices, some examples of which include but are not limited to feeding syringes, feeding tubes, feeding sets, extension devices, medication syringes, other medical devices, other feeding devices, accessories, adapters, vessels, bulbs, ampules, and the like. In examples illustrated herein, the connector 102 is a male connector configured to connect to compatible female devices. In alternative implementations, however, the connector 102 is a male connector configured to connect to female devices. In this manner, the connector 102 provides a secure, threaded design that assures a leak-proof connection to compatible devices.

In one or more implementations, the connector 102 is an ISO 80369-3 (ENFit®-type) compatible connector, and thus facilitates connection with other ENFit® products. ENFit® is the registered mark of Global Enteral Device Supplier Association, Inc., which licenses the use of its mark to others for an internationally standardized connection system that reduces the risk of misconnection by ensuring only enteral devices can connect to each other. The ENFit® system was developed by an international team of clinicians, manufacturers, and regulators (including the United States Food and Drug Administration) seeking to decrease small-bore tubing misconnections. Collaborating with the International Organization of Standardization (ISO) and Association for Advancement of Medical Instrumentation (AMMI), the group developed the ISO 80369 series of standards, with ISO 80369-3 (referred to by the tradename “ENFit®” when used under license) being the enteral standard. Although aspects of the present disclosure are described in context of ENFit-compatible devices, those skilled in the art will appreciate the applicability of alternative connectors without departing from the scope of the appended claims.

In addition to the ENFit®-type connector, the fluid administration device 100 can be implemented with other connector interfaces to enhance compatibility with various fluid delivery systems. These include oral syringe-compatible female connectors, which are commonly used in pharmaceutical dispensing to prevent misconnections with intravenous or enteral devices. The fluid administration device 100 can also include interfaces compatible with ointment-style tubes, allowing for direct connection and transfer of viscous or semi-solid formulations.

The connector 102 can be connected to a rigid, semi-rigid, or pliable tube 104 (hereafter “tube 104”) via a first tube interface 106 at the proximal end of the tube 104. The first tube interface 106 provides a passage through which fluid will travel from an attached device (e.g., a syringe, bulb, ampule, or other vessel) into a body of the tube 104. The tube 104 can be at least partially inserted into the connector 102 at the first tube interface 106 to create a leak-proof seal. In the illustrated implementation, the first tube interface 106 provides a step-down adapter that adapts the larger inside diameter of the connector 102 to the smaller outside diameter of the tube 104. Alternative implementations are contemplated to accommodate the same or different inside diameters of the connector 102 and outside diameters of the tube 104. Moreover, the illustrated implementation depicts the connector 102 including the first tube interface 106, such as part of the same unit. In alternative implementations, the first tube interface 106 is removable from the connector 102 so as to accommodate tubes 104 of varying diameters.

The first tube interface 106 can include an aperture suitable in size to accept the outer diameter of the proximal end of the tube 104. In one or more implementations, the first tube interface 106 provides a friction-fit between the connector 102 and the tube 104. In one or more implementations, a mechanical fastener, such as a barbed fitting (not shown), is used at or near the first tube interface 106 to create a leak-proof seal between the connector 102 and the tube 104. In one or more implementations, a medical grade adhesive is used to bond the connector 102 and the tube 104 at or near the first tube interface 106. In other implementations, the connector 102 and the tube 104 are manufactured as a single integral unit. In some other implementations, the connector 102 and the tube 104 can be joined using a friction fit, ultrasonic welding, thermal bonding, solvent bonding, overmolding, or mechanical interlock such as a snap-fit or twist-lock mechanism.

In general, the tube 104 can be made from a material that is resistant to breakage, even if bitten, safeguarding the tube 104 against perforating skin and tissue and making it safe for use in the buccal cavity. The length of the tube 104 can be selected to fit comfortably within the buccal cavity or for its desired application, allowing for easy maneuverability and positioning. For instance, the tube 104 can be made from a medical safe material, some example of which include but are not limited to poly-vinyl chloride (PVC), polypropylene (PP), silicone, thermoplastic elastomer, fluoropolymers, ethylene-propylene diene monomer (EPDM), other plastics, or other rubbers. The tube 104 can be made from natural materials, synthetic materials, or a combination of both. The material used to make the tube 104 can be selected based on the desired characteristics of the tube 104, such as level of purity, flexibility, kink resistant, tensile strength, chemical resistance, operating temperature range, other characteristics, and/or the like.

The body of the tube 104 allows fluid to flow from the connector 102, through the first tube interface 106 (if applicable), towards the distal end of the tube 104, through a second tube interface 108, and into a foam applicator 110. The tube 104 can be at least partially inserted into the foam applicator 110 at the second tube interface 108 to create a bonded connection. In another implementation, second tube interface 108 provides a step-up adapter that adapts the smaller outside diameter of the tube 104 to the larger inside diameter of foam applicator 110. Alternative implementations are contemplated to accommodate the same or different diameters of the tube 104 and the foam applicator 110. Moreover, the illustrated implementation depicts the foam applicator 110 including the second tube interface 108, such as part of the same unit. In alternative implementations, the second tube interface 108 is removable from the foam applicator 110 so as to accommodate tubes 104 or foam applicators 110 of varying diameters.

The second tube interface 108 can include an aperture suitable in size to accept the outer diameter of the distal end of the tube 104. In one or more implementations, the second tube interface 108 provides a friction-fit between the tube 104 and the foam applicator 110. In one or more implementations, a mechanical fastener, such as a barbed fitting (not shown), is used at or near the second tube interface 108 to create a leak-proof coupling between the tube 104 and the foam applicator 110. In one or more implementations, a medical grade adhesive is used to secure the tube 104 and the foam applicator 110 at or near the second tube interface 108. In one or more implementations, a heated bonding or ultra sonic welding process is used to secure the tube 104 and the foam applicator 110 at or near the second tube interface 108. In other implementations, the tube 104 and the foam applicator 110 are manufactured as a single integral unit. In some implementations, one or more fluid dispersion apertures can be formed near the distal end of the tube 104, underneath the foam applicator 110. These apertures can be created through molding, drilling, or thermal punching and serve to evenly distribute fluid into the foam applicator 110 during administration.

The foam applicator 110 can be designed to be non-shedding to ensure no foam particles are left behind in the mouth of patient. The foam applicator 110 allows for controlled administration of fluid, ensuring even distribution across the buccal mucosa. The porosity of the foam applicator 110 ensures the fluid is absorbed and then controllably released/administered, thereby enhancing the effectiveness of the administration. In one or more implementations, the foam applicator 110 is made from medical grade polyurethane foam, although other medical grade foam materials are contemplated.

As used herein, “controlled administration” or “controlled release” refers to the regulated delivery of a fluid from an applicator in a manner that avoids immediate expulsion and allows the fluid to be absorbed over a period of time or in a controlled fashion. This includes implementations where the fluid is initially delivered as a bolus but is retained by the applicator material—such as a porous foam—and subsequently released to the target site in a manner that promotes effective absorption, minimizes loss, or aligns with therapeutic intent. Controlled release may be influenced by characteristics of the applicator material, including porosity, capillarity, absorption capacity, and surface interaction with tissue.

The porosity of the foam applicator 110 can be selected based on the type of fluid to be administered. For instance, a 100 pores per inch (PPI) foam may be suitable for administration of medicines, colostrum, and/or other fluids. Foams having a higher or lower PPI are also contemplated. In one non-limiting example, the foam may be selected from a range of PPI values, such as approximately 80-130 PPI.

The foam applicator 110 can function not only as a passive delivery medium but also as a therapeutic interface that enables massage-based dispersion and buccal tissue stimulation, supporting both local and systemic effects. In some implementations, the foam applicator 110 includes an anti-reuse feature, such as a colorimetric change, hardening of residual medication, or textural alteration upon exposure to fluid, thereby discouraging reuse and ensuring single-use integrity.

In various implementations, the tube 104 of the fluid administration device 100 is pliable and bite-resistant, minimizing the risk of breakage when used with pediatric or special needs populations. The tube 104 can have a length optimized to reach the buccal cavity safely. The foam applicator 110 at the distal end is non-shedding and porous, allowing for controlled delivery of viscous fluids and gels. The foam structure not only holds the therapeutic substance but also facilitates massaging into the buccal mucosa, enhancing local or systemic absorption while significantly reducing the risk of spitting or expulsion of the medication. In some implementations, the foam applicator 110 can function as a passive filtration interface, filtering large particulates or undesired materials from the administered fluid. This feature enhances safety and ensures the foam applicator 110 operates not merely as a passive delivery system but as a therapeutic interface.

In some implementations, the foam applicator 110 can be flavored to increase acceptance, particularly among pediatric patients. For example, the foam applicator 110 can be pre-treated with pharmaceutical-grade flavoring agents such as grape, bubblegum, or other pediatric-friendly flavors to promote compliance and user comfort during administration.

The fluid administration device 100 supports oral therapy in unresponsive or unconscious individuals. Additionally, when administering colostrum or other biologically active fluids, the foam applicator 110 can deliver small doses while supporting cue-based feeding through lip stimulation. Use of a pre-moistened or sterile foam substrate also improves aseptic technique, contrasting with prior methods requiring manual swab wetting. In some implementations, the fluid administration device 100 is configured to couple with a pre-sealed syringe or collapsible vessel. A male nozzle on the connector 102 can be used to pierce the seal of such a container during attachment, forming an aseptic connection and allowing the transfer of fluid directly through the fluid administration device 100 and into the foam applicator 110. In some implementations, the connector 102 is adapted to interface with oral syringe-compatible female connectors or with medication containers such as ointment-style tubes.

In one or more implementations, the foam applicator 110 can be used to retain analgesic agents in the buccal cavity for localized administration, which is particularly useful in teething infants. The foam applicator 110 can hold the therapeutic fluid in place, avoiding unintentional swallowing and allowing prolonged local exposure. The foam applicator 110 can also retain viscous analgesics such as benzocaine formulations for localized buccal application during teething or mucosal pain management.

FIG. 1B is a schematic diagram depicting another orthographic side view of the fluid administration device 100 according to an example implementation. The connector 102, the tube 104, the first tube interface 106, the second tube interface 108, and the foam applicator 110 are shown in this view.

Turning now to FIG. 1C, shown is a schematic diagram depicting a perspective side view of the fluid administration device 100 according to an example implementation. The connector 102, the tube 104, the first tube interface 106, the second tube interface 108, and the foam applicator 110 are shown in this view. An interior portion of the connector 102 is shown in greater detail. For instance, the interior of the connector 102 includes a threaded section 112 having one or more threads that allow the connector 102 to be securely attached to a corresponding female connector. Although not shown in the illustrated implementation, the bottom interior portion of the connector 102 can have an internal sealing mechanism that ensures the contents do not leak when the connection is made. The internal sealing mechanism can be a rubber or silicone gasket or O-ring or can include a narrowed lumen that is resistant to the free flow of fluid.

The connector 102 also includes a fluid orifice 114 through which fluid passes into the connector 102 to be delivered through the rest of the fluid administration device 100. The fluid orifice 114 can be a standard size or selected based on the type of fluid being administered. The fluid orifice 114 or surrounding portion can include a piercing mechanism (not shown) that allows a seal to be pierced when the connector 102 is attached to a sealed syringe or other compatible device. In this manner, the syringe or compatible device can contain a single dose of medication that remains sealed until the device is attached to the connector 102. The fluid orifice 114 can include a valve to prevent backflow of fluid into the syringe, although one or more backflow valves can be implemented in the first tube interface 106, in the tube 104, in the second tub interface 108, in the foam applicator 110, or in multiple locations within the fluid administration device 100.

The connector 102 can include one or more safety and tactile features, such as wings or flanges, to facilitate handling and to further prevent accidental disconnections. In one or more implementations, once the connector 102 is attached to a syringe or other device, the wing, flange, or other safety feature ensures the connector 102 is permanently attached to the syringe and is rendered unusable (e.g., broken) if an attempt is made to detach the two parts. This provides additional security against cross contamination.

FIG. 1D is a schematic diagram depicting another perspective side view of the fluid administration device 100 according to an example implementation. The connector 102, the tube 104, the first tube interface 106, the second tube interface 108, and the foam applicator 110 are shown in this view.

Turning now to FIG. 1E, a schematic diagram is shown depicting an orthographic rear view of the fluid administration device 100 according to an example implementation. The connector 102 and the foam applicator 110 are shown in this view.

FIG. 1F is a schematic diagram depicting an orthographic front view of the fluid administration device 100 according to an example implementation. The connector 102, the threaded section 112, and the fluid orifice 114 are shown in this view.

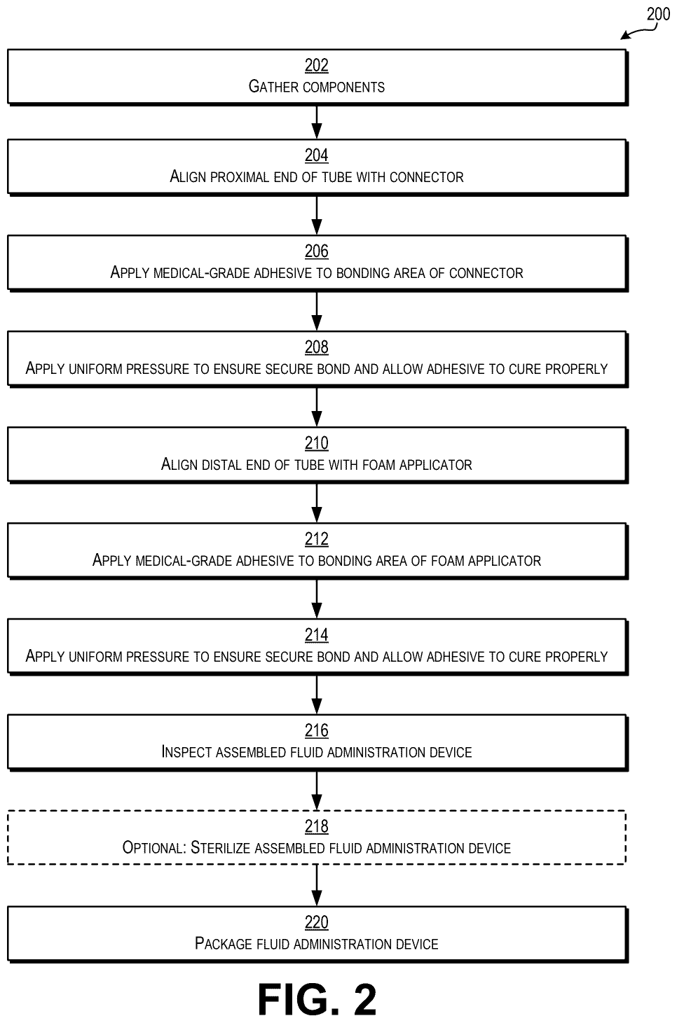

Turning now to FIG. 2, a flow diagram is shown depicting a method 200 for assembling the fluid administration device 100 according to an example implementation. It should be understood that the operations of the methods disclosed herein are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the appended claims.

The method 200 begins and proceeds to block 202. At block 202, the various components of the fluid administration device 100 are gathered, including the connector 102, the tube 104, and the foam applicator 110. These components may be sterilized as standalone components and sterilized again after assembly or assembly and components may remain non-sterilized.

In implementations that utilize a separate first tube interface 106 and/or a separate second tube interface 108, these components can be pre-assembled with their respective counterparts—the connector 102 to the first tube interface 106 and the foam applicator 110 to the second tube interface 108, or some alternative configuration. For ease of explanation of the method 200, the connector 102, the tube 104, and the foam applicator 110 are the three components gathered for assembly. Any adapter(s) provided by the first tube interface 106 and/or the second tube interface 108 is/are built-in as described above in some example implementations.

At block 204, the proximal end of the tube 104 is aligned with the connector 102. At block 206, a medical-grade adhesive is applied to a bonding area of the connector 102. The bonding area may be a predesignated area that has been prepared (e.g., as part of a manufacturing process) to receive the medical-grade adhesive. The bonding area can have a different texture to promote adhesion. The bonding area can be pretreated with an accelerant designed to expedite the adhesive curing process. The bonding area can be marked (e.g., with permanent non-toxic marker) or may be differentiated in color or some other manner. The bonding area can have a pre-applied adhesive with a protective layer preventing the adhesive from curing prior to being removed and exposed, for instance, to oxygen or water which initiates the curing process. Alternatively, the tube 104 can be secured using a friction fit, barbed fitting, ultrasonic welding, thermal bonding, solvent bonding, or another medically acceptable mechanical or chemical fastening technique.

At block 208, uniform pressure is applied to ensure a secure bond and allow the adhesive to cure properly. The pressure can be applied for a minimum set time (e.g., 15-30 second) and then be allowed to continue curing. Alternatively, the pressure can be applied until the adhesive is fully cured. The total curing time depends upon the adhesive used, ambient temperature, humidity, and/or other environmental factors.

At block 210, the distal end of the tube 104 is aligned with the foam applicator 110. At block 212, a medical-grade adhesive is applied to a bonding area of the foam applicator 110. The bonding area can be a predesignated area that has been prepared (e.g., as part of a manufacturing process) to receive the medical-grade adhesive. The bonding area can have a different texture to promote adhesion. The bonding area can be pretreated with an accelerant designed to expedite the adhesive curing process. The bonding area can be marked (e.g., with permanent non-toxic marker) or may be differentiated in color or some other manner. The bonding area can have a pre-applied adhesive with a protective layer preventing the adhesive from curing prior to being removed and exposed, for instance, to oxygen or water which initiates the curing process. Alternative securing methods can include mechanical friction fits, barbed insertions, ultrasonic welding, or thermal bonding.

At block 214, uniform pressure is applied to ensure a secure bond and allow the adhesive to cure properly. The pressure can be applied for a minimum set time (e.g., 15-30 second) and then be allowed to continue curing. Alternatively, the pressure can be applied until the adhesive is fully cured. The total curing time depends upon the adhesive used, ambient temperature, humidity, and/or other environmental factors.

At block 216, the assembled fluid administration device 100 is inspected for any defects or irregularities. For instance, the bonds at both the connector 102 and the foam applicator 110 can be checked to ensure these bonds are secure and free from gaps. Additionally, the tube 104 can be checked for pliability and to ensure it is free from any imperfections or weak points that could compromise its integrity or usability.

At block 218, optionally, the assembled fluid administration device 100 is sterilized using an appropriate sterilization method, such as ethylene oxide (EtO) gas sterilization or gamma irradiation, ensuring it meets all relevant medical standards for sterility. At block 220, the fluid administration device 100 is individually packaged in a sterile, airtight package to maintain sterility until use. In addition, the packaging can be labeled with relevant information, including the product name, lot number, sterilization date, and expiration date as applicable.

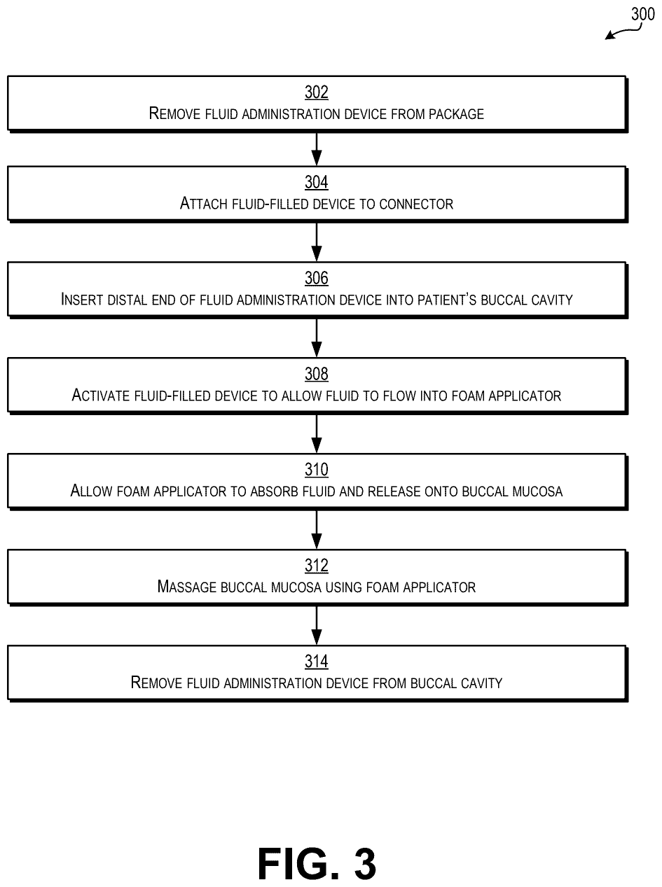

Turning now to FIG. 3, a flow diagram is shown depicting a method 200 for using the fluid administration device 100 according to an example implementation. The method 300 begins and proceeds to block 302. At block 302, the fluid administration device 100 is removed from its package. At block 304, a fluid-filled device, such as a syringe or compatible product with medication or therapeutic fluid, is attached to the connector 102 on the proximal end of the fluid administration device 100 to ensure a secure and leak-proof connection. In one or more implementations, the fluid-filled device is a single use device that is factory-filled with a specific amount of fluid, such as a single dose of medication. Upon attaching the fluid-filled device to the connector 102, a seal can be pierced allowing the fluid inside to flow into the fluid administration device 100.

At block 306, the distal end of the fluid administration device 100 is inserted into a patient's buccal cavity. The supple nature of the tube 104 ensures comfort and reduces the risk of injury to the patient during insertion.

At block 308, the fluid-filled device is activated, allowing the fluid to flow through the tube 104 and into the foam applicator 110. For instance, if the fluid-filled device is a syringe, a plunger can be slowly depressed thereby forcing the fluid from the barrel of the syringe into the fluid administration device 100. Alternative activation mechanisms are contemplated, some examples of which include but are not limited to pressure-based mechanisms, buttons, gravity-fed mechanisms, and so on.

At block 310, the foam applicator 110 is allowed to absorb fluid and subsequently release the fluid in a controlled manner onto the buccal mucosa. In some cases, the foam applicator 110 may be allowed to absorb fluid for a minimum contact time before applying the foam applicator 110 to the buccal mucosa. This controlled release ensures efficient and consistent absorption of the medication or therapeutic fluid.

At block 312, the foam applicator 110 is used to massage the buccal mucosa. This enhances absorption and ensures the fluid is evenly distributed across the buccal mucosa. At block 314, after the desired amount of fluid has been delivered and absorbed, the fluid administration device 100 is carefully removed from the patient's buccal cavity and discarded.

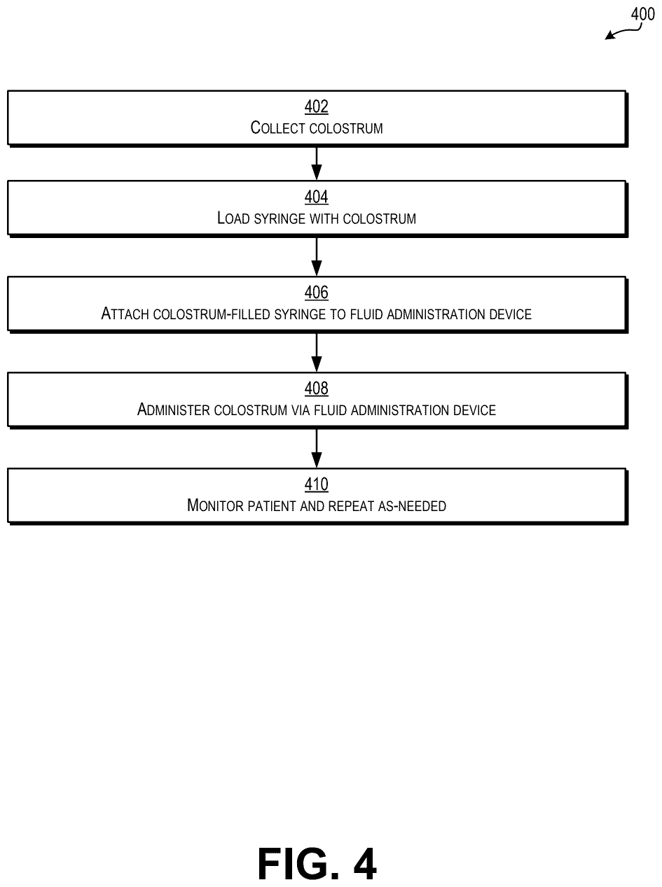

Turning now to FIG. 4, a flow diagram is shown depicting a method 400 for colostrum therapy and the prophylactic administration of colostrum via the fluid administration device 100 according to an example implementation. Colostrum is produced by mothers in the first few days after giving birth. Colostrum is rich in immunoprotective and anti-infective agents, as well as growth factors such as immunoglobulin A, cytokines, lysozymes, lactoferrin, and epidermal growth factor. These components protect infants from infection, stimulate gastrointestinal development, and modulate the immune system, particularly in preterm infants. Preterm colostrum is especially potent, containing higher concentrations of immunologically active factors compared to term colostrum. Buccal administration of colostrum can provide multiple benefits, such as stimulating the oropharyngeal-associated lymphoid tissue system, systemic absorption of protective factors through the buccal mucosa, acting as a barrier to prevent microbial adhesion, and enhancing gastrointestinal development. The method 400 is particularly beneficial in reducing the risk of late-onset sepsis (LOS), necrotizing enterocolitis (NEC), and ventilator-associated pneumonia in preterm infants.

The method 400 begins and proceeds to block 402. At block 402, colostrum is collected from the mother using one or more of various techniques. For instance, the colostrum can be collected manually or with a breast pump. The collected colostrum can be stored in a container and labeled with the mother's identifying information, date, and time of collection to maintain accurate records.

At block 404, the colostrum is loaded into a syringe or other vessel such as a dropper or an ampule. A small amount of colostrum may be used. For instance, a typical amount of colostrum is between 0.2 and 0.5 milliliters (mL). At block 406, the colostrum-filled syringe or vessel is attached to the connector 102 on the proximal end of the fluid administration device 100.

At block 408, the colostrum is administered via the fluid administration device 100. For instance, the distal end of the fluid administration device 100 having the foam applicator 110 is inserted into the patient's buccal cavity and positioned against the buccal mucosa. The syringe plunger is then slowly depressed to release the colostrum into the foam applicator 110. The foam applicator 110 absorbs the colostrum and allows for a controlled release of the colostrum onto the buccal mucosa. At block 410, the patient can be monitored, and the method 400 can be repeated as-needed to administer more colostrum.

By utilizing the fluid administration device 100, the administration of colostrum to preterm and critically ill infants is enhanced through a controlled and hygienic process. The fluid administration device 100 ensures that colostrum is delivered effectively into the buccal cavity, optimizing the absorption of its immunoprotective and growth factors. The method 400 leverages the unique benefits of buccal administration, providing a safe and effective prophylactic measure against sepsis, NEC, and other complications, ultimately supporting the health and development of vulnerable infants.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”. As used herein, the term “approximately” is intended to account for variations that are acceptable to a person of ordinary skill in the art and that do not materially affect the intended function or performance of the component, measurement, or value being described. The scope of “approximately” may vary depending on context, but generally includes deviations within a range that achieve substantially the same result.

The features, structures, or characteristics described above may be combined in one or more embodiments in any suitable manner, and the features discussed in the various embodiments are interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims.

The terms “first,” “second,” etc. are used only as labels, rather than a limitation for a number of the objects. It is understood that if multiple components are shown, the components may be referred to as a “first” component, a “second” component, and so forth, to the extent applicable.

Disjunctive language, such as the phrase “at least one of X, Y, or Z,” unless specifically stated otherwise, is to be understood with the context as used in general to present that an item, term, etc., can be either X, Y, or Z, or any combination thereof (e.g., X, Y, and/or Z). Thus, such disjunctive language is not generally intended to, and should not, imply that certain embodiments require at least one of X, at least one of Y, or at least one of Z to be each present.

The above-described implementations of the present disclosure are merely possible examples set forth for a clear understanding of the principles of the disclosure. Many variations and modifications may be made to the above-described implementation(s) without departing substantially from the spirit and principles of the disclosure. All such modifications and variations are intended to be included herein within the scope of this disclosure and protected by the following claims.

Therefore, the following is claimed:

1. An apparatus for delivering a fluid, the apparatus comprising:
a connector configured to connect to a corresponding compatible device;
a tube having a proximal end and a distal end, the proximal end affixed to the connector; and
an applicator connected to the distal end of the tube, wherein the applicator is configured to receive the fluid from the tube and deliver the fluid to a target site.

2. The apparatus of claim 1, wherein the connector is an International Organization for Standardization (ISO) 80369-3 male connector.

3. The apparatus of claim 1, wherein the connector is configured to pierce a sealed portion of the corresponding compatible device.

4. The apparatus of claim 3, wherein the corresponding compatible device comprises a prefilled syringe or a sealed fluid reservoir.

5. The apparatus of claim 1, wherein the connector is configured to interface with an oral syringe-compatible female connector.

6. The apparatus of claim 1, wherein the tube is pliable and bite-resistant.

7. The apparatus of claim 1, wherein the tube comprises a plurality of fluid dispersion apertures adjacent to the distal end of the tube.

8. The apparatus of claim 1, wherein the tube is formed from a medical-grade polymer, the medical-grade polymer comprising polyvinyl chloride, polypropylene, silicone, thermoplastic elastomer, or ethylene-propylene diene monomer (EPDM).

9. The apparatus of claim 1, wherein the applicator comprises a non-shedding, porous foam.

10. The apparatus of claim 9, wherein the non-shedding, porous foam has a porosity in a range of approximately 80 to 130 pores per inch (PPI).

11. The apparatus of claim 1, wherein the applicator is configured to deliver the fluid to a patient's buccal mucosa.

12. The apparatus of claim 1, wherein the applicator comprises a feature configured to indicate prior use, the feature comprising a color change or a material hardening.

13. The apparatus of claim 1, wherein the applicator comprises a flavored substrate.

14. The apparatus of claim 1, wherein the applicator retains an analgesic for prolonged local exposure to a mucosal surface.

15. A method for assembling an apparatus for delivering a fluid, the method comprising:
aligning a proximal end of a tube with a connector configured to couple with a vessel comprising the fluid;
securing the proximal end of the tube to the connector;
aligning a distal end of the tube with an applicator; and
securing the distal end of the tube to the applicator.

16. The method of claim 15, wherein the applicator is configured to retain the fluid and provide a controlled administration the fluid.

17. The method of claim 15, wherein the distal end of the tube comprises a plurality of apertures configured to disperse the fluid into the applicator.

18. The method of claim 15, wherein the applicator comprises a foam material having a porosity selected to match a viscosity of the fluid.

19. The method of claim 15, wherein the applicator comprises a flavoring agent selected to enhance patient acceptance.

20. The method of claim 15, wherein the applicator is configured to deliver the fluid to a buccal surface.

21. The method of claim 15, further comprising integrating an indicator feature into the applicator to visually signal prior use.

22. The method of claim 15, wherein the assembled apparatus is configured for delivery of a colostrum composition.

23. A system for delivering a fluid, the system comprising:
a vessel containing the fluid; and
an apparatus comprising:
a connector configured to couple with the vessel;
a tube having a proximal end secured to the connector and a distal end; and
an applicator secured to the distal end of the tube and configured to receive the fluid from the vessel.

24. The system of claim 23, wherein the vessel comprises a prefilled syringe.

25. The system of claim 23, wherein the applicator is configured to release the fluid onto a buccal surface.

26. The system of claim 23, wherein the connector comprises a male fitting configured to pierce a sealed outlet of the vessel.

27. The system of claim 23, wherein the applicator comprises a porous foam material with a porosity selected to match a viscosity of the fluid.

28. The system of claim 23, wherein the vessel contains colostrum.

29. The system of claim 23, wherein the applicator comprises a flavoring agent.

30. The system of claim 23, wherein the vessel and the apparatus are packaged together in a sterile container.
